(19) United States
(12) Plant Patent Application Publication
(10) Pub. No.: US 2025/0248321 P1
(43) Pub. Date: Jul. 31, 2025

(54) SALVIA PLANT NAMED 'DH002'

(50) ;

(71) Applicant: Desert Honed LLC, Boise, ID (US)

(72) Inventor: Nicholas A. Boynton-Steele, Boise, ID (US)

(73) Assignee: Desert Honed LLC, Boise, ID (US)

(21) Appl. No.: 19/037,198

(22) Filed: Jan. 25, 2025

Related U.S. Application Data
(60) Provisional application No. 63/627,192, filed on Jan. 31, 2024.

(51) Int. Cl. A01H 6/50

(52) U.S. Cl. CPC A01H 6/508
USPC PLT/475

(57) ABSTRACT

A new Salvia plant designated ‘DH002’ particularly distinguished by greyed-green pubescent leaves with a shrub height of 70 cm, is disclosed.

### CROSS REFERENCE TO RELATED MATTER

The present application claims priority to U.S. application Ser. No. 63/627,192, as filed on Jan. 31, 2024, the entire contents of which is incorporated herein by reference for all purposes.

Genus and species: Salvia hybrida.

Variety denomination: ‘DH002’.

### BACKGROUND OF THE NEW PLANT

The present invention comprises a new and distinct variety of Salvia, botanically known as Salvia hybrida, and hereinafter referred to by the variety name ‘DH002’. ‘DH002’ originated from a cross of the proprietary female Salvia plant ‘1603-5-T2’ (unpatented) and the proprietary male Salvia plant ‘1200-6-P1’in June 2018 in Tucson, Arizona.

In fall of 2018 seeds were planted and a plant named ‘DH002’ was identified in the summer of 2019. ‘DH002’ was first vegetatively propagated by vegetative cuttings in Boise, Idaho. ‘DH002’ was found to reproduce true to type in successive generations of asexual propagation in Boise, Idaho via vegetative cuttings.

### SUMMARY OF ‘DH002’

The following are the most outstanding and distinguishing characteristics of ‘DH002’ when grown under normal horticultural practices in Boise, Idaho.
1. Greyed-green pubescent leaves; and
2. Shrub height of 70 cm.

### DESCRIPTION OF THE PHOTOGRAPHS

These new Salvia plants are illustrated by the accompanying photographs of plants grown in a greenhouse in Boise, Idaho. The colors shown are as true as can be reasonably obtained by conventional photographic procedures.

FIG. 1 is a whole plant photo of ‘DH002’ showing foliage, buds, and flowers. The photo is of a 2-year-old plant taken in June 2021 in Boise, Idaho.

FIG. 2 is close-up photo of ‘DH002’ showing foliage, buds, and flowers. The photo is of a 1-year-old plant taken in June 2020 in Boise, Idaho.

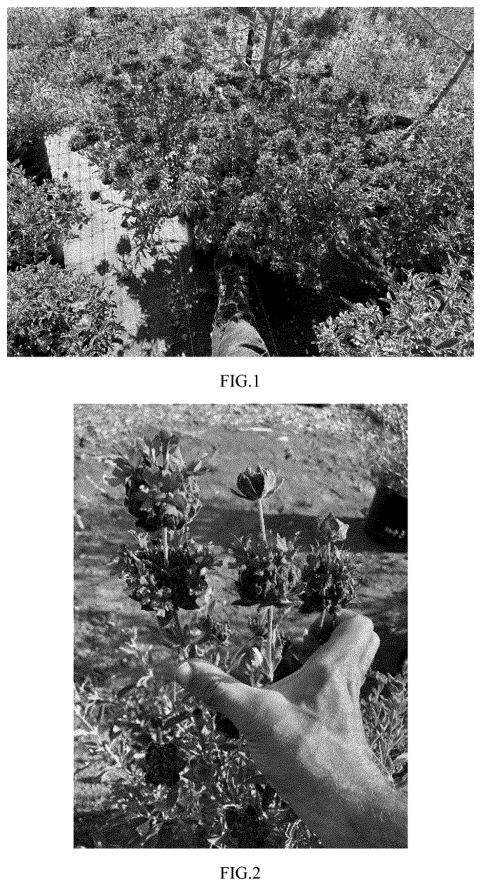

### DESCRIPTION OF ‘DH002’

The following detailed descriptions set forth the distinctive characteristics of ‘DH002’.The data which define these characteristics were collected from asexual reproductions carried out in Boise, Idaho. Data was collected in January 2024 on one year old plants from cuttings in 3gallon containers grown in a plastic greenhouse. Color references are to The R.H.S. Colour Chart of The Royal Horticultural Society of London (R.H.S.), 4^{th} edition (2001).
Classification:
   Family.—Lamiaceae.
   Genus.—Salvia.
   Species.—hybrida.
   Common name.—Sage.
Parents:
   Female.—Salvia plant ‘1603-5-T2’ (unpatented).
   Male.—Salvia plant ‘1200-6-P1’ (unpatented).
Plant:
   Propagation method.—Cutting.
   Rooting system.—Fibrous.
   Vigor.—Medium.
   Time to develop roots.—2 to 3 weeks.
   Crop time.—2 months to finish a one gallon, 3 to 4 months to finish a 3 gallon.
   Suggested container size.—One, three or five gallon containers.
   Use.—Home gardens, commercial landscapes, native gardens, waterwise gardens, pollinator gardens.
   Type.—Perennial.
   Overall dimensions.—70 cm tall×100 cm wide.
   Cultural requirements.—In ground, full to partial sun, infrequent deep watering, light shearing after main bloom season will keep the shrub compact. In containers — Full sun, well drained media, consistently moist, pinched in plug stage, pinched at least once more for a one gallon, at least twice more for a 3 or 5 gallon.
   Hardiness.—USDA zone 8a.
   Form.—Low, rounded shrub.
   Growth habit.—Compact, rounded.
   Blooming season.—Late spring through summer.
   Lastingness of blooms.—Each flower 2 days, each inflorescence 4 to 6 weeks. Dried glomerules last for months.
Stems (below first pinch):
   Condition.—Semi-ripe wood.
   Bark.—Lightly tomentose becoming rough.
   Shape.—Square in cross section becoming round.
   Length.—6 cm to 8 cm.
   Diameter.—0.3 cm.
   Bark color.—RHS 165A and RHS 166C.
   Stem color (where exposed beneath bark).—RHS 193A.
   Shape.—Square in cross section becoming round.
   Texture.—Lightly tomentose becoming rough.
Branches:
   Quantity.—30 to 50.
   Shape.—Square in cross section.
   Length.—8 cm to 12 cm.
   Width.—0.15 cm to 0.2 cm.
   Internode length.—3.8 cm to 0.2 cm. Spring growth has larger leaves and longer internode lengths.
   Color.—RHS 187 A at branch tips, RHS 194B in middle, then fading to RHS 165A at base.
   Anthocyanin.—Absent.
Leaves:
   Type, arrangement.—Opposite.
   Attachment.—Petiolate.
   Quantity per branch.—8 to 16.
   Shape.—Ovate to elliptical.
   Length (mature).—3.0 cm to 5.3 cm.
   Width (mature).—1.2 cm to 1.8 cm.
   Margin.—Crenate.
   Thickness.—0.05 cm.
   Surface (adaxial).—Rugose.
   Surface (abaxial).—Net-veined, slightly pubescent.
   Color (adaxial surface).—RHS 189A.
   Color (abaxial surface).—RHS 191B.
   Venation.—Pinnate in pattern, recessed from upper surface, protruding from lower surface.
   Vein color.—RHS 194B.
   Apex.—Acute to rounded.
   Base.—Moderately taped to petiole.
   Fragrance.—Musk, slight cineole.
   Stipules.—Absent.
Petiole:
   Shape.—D-shaped in cross section.
   Length.—1.0 cm to 1.4 cm.
   Width.—0.1 cm.
   Texture.—Tomentose.
   Color.—RHS 194C.
Disease and pest susceptibility: Occasional Powdery Mildew. Occasional Pithium or other water mold susceptibility in water-logged or poorly drained soil. Occasional aphid infestation in early spring on new growth.

### COMPARISON OF ‘DH002’ WITH PARENTAL LINES AND KNOWN VARIETY

‘DH002’ is distinguished from the female parent as described in Table 1 and the male parent in Table 2.

TABLE 1
Comparison with Female Parental Line

| | Variety | Female parent |
|---|---|---|
| Trait | ‘DH002’ | ‘1603-5-T2’ |
| Leaf pubescence | Pubescence is | Foliage lacks pubescence |
| | present on | |
| | foliage | |

TABLE 2
Comparison with Male Parental Line

| | Variety | Male Parent |
|---|---|---|
| Trait | ‘DH002’ | ‘1200-6-P1’ |
| Adaxial Leaf color | RHS 189A | RHS 190A with |
| | | indumentum and RHS |
| | | 191A under |
| | | indumentum. |

‘DH002’ can be compared to the commercial Salvia variety ‘Allen Chickering’ (unpatented). Differences between the varieties are described in Table 3.

TABLE 3
Comparison with Similar Variety

| | Variety | Commercial line |
|---|---|---|
| Trait | ‘DH002’ | ‘Allen Chickering’ |
| Height | 70 cm | 130 cm to 160 cm |

I claim:

1. A new and distinct variety of Salvia plant named ‘DH002’ as shown and described herein.
